(19) United States
(12) Patent Application Publication
(10) Pub. No.: US 2017/0000392 A1
(43) Pub. Date: Jan. 5, 2017

(54) Micro-Camera Based Health Monitor

(71) Applicant: Rememdia LC, Salt Lake City, UT (US)

(72) Inventor: Fraser Smith, Salt Lake City, UT (US)

(21) Appl. No.: 14/789,732

(22) Filed: Jul. 1, 2015

(51) Int. Cl. A61B 5/1455; A61B 5/00; A61B 5/01; A61B 5/021; A61B 5/08

(52) U.S. Cl. CPC A61B 5/14551; A61B 5/02141; A61B 5/0816; A61B 5/015; A61B 5/6824; A61B 2576/00

(57) ABSTRACT

A camera coupled to a processor is disclosed. The camera is configured to capture images of the subject. The processor is configured to amplify microscopic temporal variations between the images of the subject and generate a profile of at least one microscopic temporally detected physiological variation of the tissues of the subject. The processor is further configured to compare the profile of the subject to a pre-existing profile of the subject and/or an aggregate profile of a plurality of third-party subjects, said aggregate profile corresponding to the at least one microscopic temporally detected physiological variation of the third-party subjects, the aggregate third-party profile corresponding to a known state of the tissue of the third-party subjects.

### FIELD OF THE TECHNOLOGY

The present technology relates to improved devices, methods, and systems for monitoring the health of a subject. More particularly, the present technology relates to devices, methods, and systems for assessing the condition or health state of living tissues of a subject.

### BACKGROUND OF THE TECHNOLOGY

The increasing complexity of healthcare is causing fragmentation of care compromising patient safety and hospital efficiency. Increased costs of healthcare corresponding to the volumes of data and difficulty in assessing the state of the patient compound problems associated with patient safety and efficient treatment. Many treatment options and diagnoses, however, are made as the result of acute conditions or conditions that are readily observable to the medical practitioner during an office visit precipitated by an acute medical event. It is believed that many illnesses, ailments, health conditions, etc., including serious illnesses (e.g., cancer, leukemia, etc.), can be detected before they warrant significant medical attention and/or before significant adverse affects or symptoms are felt by the subject.

### SUMMARY OF THE INVENTION

In light of the problems and deficiencies inherent in the prior art, the present invention seeks to overcome these by providing methods, devices, and systems configured to measure variations present in one or more tissues of a subject and correlate those variations to disease states or healthy states of tissues for pre-treatment of a condition, treatment of a condition without an acute medical event, and/or for treatment prior to more intrusive examination and/or excising/sampling of tissues. In one example discussed herein, a wearable device is configured to monitor a physiological condition of a subject, comprising a wearable camera having a coupling member configured to attach the camera to a portion of the subject, the camera configured to capture a plurality of images of a portion of the tissue of the subject. A processor is in communication with the camera, the processor comprising executable code configured to amplify microscopic temporal variations between the plurality of images of the tissue of the subject and generate a profile of at least one microscopic temporally detected physiological variation of the tissue of the subject and store the profile in a database. The processor is further configured to compare the profile of the tissue of the subject with a database corresponding to previous profiles of the at least one microscopic temporally detected physiological variation of the tissue of the subject.

In one aspect of the technology, a device is configured for in-vivo monitoring of the tissue of a subject, comprising an elongate medical device configured for placement into a portion of a body of the subject. A camera is disposed about a distal end of the elongate medical device, the camera being configured to capture a plurality of images of tissue within the body of the subject. A processor is coupled to the camera and comprises executable code configured to amplify microscopic temporal variations between the plurality of images of the tissue of the subject and generate a profile of at least one microscopic temporally detected physiological variation of the tissue of the subject. The processor is further configured to compare the profile of the tissue of the subject to an aggregate profile of a first plurality of third-party subjects, said aggregate profile of the first plurality of third-party subjects corresponding to the at least one microscopic temporally detected physiological variation of tissues of the first plurality of third-party subjects, the tissues of the first plurality of third-party subjects having a normal health state. The processor is further configured to compare the profile of the tissue of the subject to an aggregate profile of a second plurality of third-party subjects, said aggregate profile of the second plurality of third-party subjects corresponding to the at least one microscopic temporally detected physiological variation of tissues of the second plurality of third-party subjects, the tissues of the second plurality of third-party subjects having a known diseased state. The processor is further configured to detect differences between the profile of the tissue of the subject and the aggregate profile of the first plurality of third-party subjects and the aggregate profile of the second plurality of third-party subjects and determine a probability that a state of the subject's tissue corresponds to the diseased state of the tissues of the second plurality of third-party subjects.

In one aspect of the technology, a non-destructive method for predicting diseased states of live tissues through optical measurements is disclosed comprising positioning a camera about an area of live tissue of a subject, wherein said camera is in communication with a processor configured to receive and process image data of the tissue. The processor comprises executable code configured to amplify microscopic temporal variations between a plurality of images of the tissue and generate a profile of at least one microscopic temporally detected physiological variation of the tissue. Image data of the tissue is received through the camera. Micro-temporal variations between the plurality of images are amplified and a profile of at least one microscopic temporally detected physiological variation of the tissue is generated. The method further comprises comparing the profile of the live tissue to an aggregate profile of a first plurality of live tissues of third-party subjects. The aggregate profile of the first plurality of the third-party subjects corresponds to the at least one microscopic temporally detected physiological variation of live tissues of the first plurality of third-party subjects. The live tissues of the first plurality of third-party subjects have a normal health state. The method further comprises the step of comparing the profile of the live tissue to an aggregate profile of a second plurality of live tissues of third-party subjects. The aggregate profile of the second plurality of third-party subjects corresponds to the at least one microscopic temporally detected physiological variation of live tissues of the second plurality of third-party subjects. The live tissues of the second plurality of third-party subjects has a known diseased state. The method further comprises determining a probability that the live tissue of the subject corresponds to the diseased state of the live tissues of the second plurality of third-party subjects.

### BRIEF DESCRIPTION OF THE DRAWINGS

The present technology will become more fully apparent from the following description and appended claims, taken in conjunction with the accompanying drawings. Understanding that these drawings merely depict exemplary aspects of the present technology they are, therefore, not to be considered limiting of its scope. It will be readily appreciated that the components of the present technology, as generally described and illustrated in the figures herein, could be arranged and designed in a wide variety of different configurations. Nonetheless, the technology will be described and explained with additional specificity and detail through the use of the accompanying drawings in which:

FIG. 1 is a flow chart illustrating aspects of the current technology; and

FIG. 2 is a plurality of diagrams illustrating aspects of the current technology.

### DETAILED DESCRIPTION OF EXEMPLARY ASPECTS OF THE TECHNOLOGY

The following detailed description of exemplary aspects of the technology makes reference to the accompanying drawings, which form a part hereof and in which are shown, by way of illustration, exemplary aspects in which the technology can be practiced. While these exemplary aspects are described in sufficient detail to enable those skilled in the art to practice the technology, it should be understood that other aspects can be realized and that various changes to the technology can be made without departing from the spirit and scope of the present technology. Thus, the following more detailed description of the aspects of the present technology is not intended to limit the scope of the technology, as claimed, but is presented for purposes of illustration only and not limitation to describe the features and characteristics of the present technology, to set forth the best mode of operation of the technology, and to sufficiently enable one skilled in the art to practice the technology. Accordingly, the scope of the present technology is to be defined solely by the appended claims. The following detailed description and exemplary aspects of the technology will be best understood by reference to the accompanying drawings and description, wherein the elements and features of the technology are designated by numerals throughout the drawings and described herein.

As used in this specification and the appended claims, the singular forms “a,” “an” and “the” include plural referents unless the context clearly dictates otherwise. Thus, for example, reference to “a layer” includes a plurality of such layers.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps can be performed, and certain of the stated steps can possibly be omitted and/or certain other steps not described herein can possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. Objects described herein as being “adjacent to” each other can be in physical contact with each other, in close proximity to each other, or in the same general region or area as each other, as appropriate for the context in which the phrase is used.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness can in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element can still actually contain such item as long as there is no measurable effect thereof.

As used herein, the term “about” is used to provide flexibility to a range endpoint by providing that a given value can be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.”

An initial overview of technology is provided below and specific technology is then described in further detail. This initial summary is intended to aid readers in understanding the technology more quickly, but is not intended to identify key or essential features of the technology, nor is it intended to limit the scope of the claimed subject matter.

Broadly speaking, the technology described herein resides in a device configured to non-destructively monitor micro-visible physiological changes in the tissues of a subject. A camera is coupled to a processor and configured to capture a plurality of images of the tissues of a subject. The processor comprises executable code configured to amplify microscopic temporal variations between the plurality of images and generate a profile of at least one microscopic temporally detected physiological variation of the tissues (e.g., minor variations in color or size or texture of the tissues). The term “microscopically temporally detected physiological variations” refers generally to very small changes between values of the same pixels from one frame of an image to the next frame over a period of time, depending on the time between frames. The changes are difficult to detect with the naked or unaided eye but nevertheless present. The variations are intended to be correlated with known states of tissue (e.g., healthy or diseased) from third-party data. The processor is further configured to compare the profile of the living tissue of a subject to a pre-existing aggregate pathology profile of a plurality of third-party subjects. The aggregate profile of the third-party subjects corresponds to the at least one microscopic temporally detected physiological variation of the principal subject. The physiological variation corresponds to a known state of the third-parties tissue. A process is employed to determine the probability that the state of the tissue of the principal subject is similar to (or dissimilar to) the known state of the tissue from the third-parties. For example, if the variations of the tissue being examined relates to the correlation between tissue coloration and/or blood flow through tissue, the aggregate third-party profile compared to the subject's tissue profile is an aggregate profile of tissue coloration and/or blood flow through tissues of third-parties having a known healthy and/or diseased state of the tissue. The processor is configured to detect differences and/or similarities between the profile of the subject and the aggregate profile of the plurality of third-party subjects and correlate the similarities and/or differences between the two to determine a probability that the subject's tissue is in a healthy state, a diseased state, or an at-risk state for development of a disease.

In one aspect, a baseline state of the subject's tissue is determined based on a plurality of measurements taken of the tissue of the subject. For example, if a particular area of skin of a patient appears abnormal and a medical practitioner is concerned that it can become cancerous and/or metastasize, the medical practitioner would capture a plurality of video images and develop a baseline of the area of skin of the patient for future analysis. Existing camera assets of the subject can be used in the current technology. For example, the subject can take or have taken a plurality of pictures of himself/herself throughout the day and include the images in the process described herein. The processor is configured to compare a current profile of the subject to the baseline profile. It is also configured to compare the current profile to a pre-existing second aggregate profile of a plurality of third-party subjects, wherein said second aggregate profile of a plurality of third-party subjects corresponds to a diseased and/or healthy state of the tissue of the plurality of third-party subjects. The profiles (of the subject and/or third-parties) are stored in a database that is modifiable and updatable with new data. That is, the aggregate profile is modified by each individual measurement of a subject's tissue. In one aspect, however, the aggregate profile used in a comparison between a current subject can be time delimited and/or can exclude historical data from the same subject. For example, the aggregate profile used in a comparison between tissues of third-parties and the tissue of the subject may not be modified by current subject measurements or, alternatively, any measurements taken of the subject within a predetermined previous time period (e.g., 1 day, 1 week, or 1 month, etc.).

### Amplification of Microscopic Variations of Tissues

In accordance with one aspect of the technology, a method is employed to analyze images of a subject's living tissue and amplify physiological variations (e.g., color, shape, size, texture, etc.) in tissues over discrete periods of time (e.g., 0.001 to 0.1 seconds, 0.1 to 1 seconds, 1 to 2 seconds, 2 to n seconds, etc.). In one aspect, a time series of color values at any spatial location (e.g., a pixel) of images of a subject are taken and microscopic variations are amplified in a given temporal frequency band of interest. The processor (or user) selects and then amplifies a band of temporal frequencies including, as one non-limiting example, the flow of blood through tissues of the body. The amplification reveals the variation of color (or lack thereof) as blood flows through the tissues. Lower spatial frequencies are temporally filtered to allow a subtle input signal to rise above the camera sensor and quantization noise. The temporal filtering approach amplifies color variation, and also reveals low-amplitude motion. For example, the method can amplify minor changes in the size or shape of a cyst or other tissue formation through which blood can flow and/or that moves in response to stimuli. The method can identify one or more tissue monuments (e.g., moles, vessels, bones, organs, etc.) in order to properly assess micro-variations in the size, shape, etc. of tissues during different image capturing events. In this manner, the tissue monuments are used as reference points to properly evaluate camera positioning or artifacts associated with positioning the tissue in different positions with respect to the camera at different image capturing events.

In addition, a false coloring module can be employed to further distinguish between the degree of amplified microscopic variations in coloration in order to further distinguish between changes in color. For example, where it is desired that differences between the microscopic variations in the coloration of tissue be more easily identifiable, color variations that occur more rapidly than other variations can be presented in a blue color (or any other contrasting color to the tissue). Moreover, if color variations between adjacent pixels are subtle (e.g., different shades of red, etc.), pixels falling in a certain pre-defined threshold, for example between 620 and 640 nm, can be decreased to below 500 nm in order to distinguish the pixels adjacent to the pixels having values greater than 640 nm. In this manner, tissues in a diseased state that have only minor color variations (even in the amplified state) from surrounding tissues can be more easily distinguished and identified.

The method's mathematical analysis employs a linear approximation related to the brightness constancy assumption used in optical flow formulations. The method also derives the conditions under which the approximation holds. This leads to a multiscale approach to magnify motion without feature tracking or motion estimation. The method studies and amplifies the variation of pixel values over time, in a spatially-multiscale manner. The Eulerian approach (i.e., the approach described herein) to motion magnification does not explicitly estimate motion, but rather exaggerates motion by amplifying temporal color changes at fixed positions. The method employs differential approximations that form the basis of optical flow algorithms.

In one aspect, the method employs localized spatial pooling and bandpass filtering to extract and reveal visually the signal corresponding to motion. This primal domain analysis allows amplification and visualization of the pulse signal at each location on the tissues of a subject, for example. Nearly invisible changes in a dynamic environment can be revealed through Eulerian spatio-temporal processing of standard monocular video sequences. The method can be run in real time. A single image framework can amplify both spatial motion and purely temporal changes (e.g., a heart pulse) and can be adjusted to amplify particular temporal frequencies.

In one aspect of the technology, a spatial decomposition module of a system first decomposes input images into different spatial frequency bands, then applies the same temporal filter to the spatial frequency bands. The outputted filtered spatial bands are then amplified by an amplification factor, added back to the original signal by adders, and collapsed by a reconstruction module to generate the output images. The temporal filter and amplification factors can be tuned to support different applications. The output images can correlate to specific numerical values related to a base or “healthy” state of tissue as well as a modified or “diseased” state of tissue. For example, a baseline determination of the blood flow of a subject's tissue under a “healthy” set of circumstances can be measured and later compared with the blood flow of the subject's tissue under a varied set of circumstances. The comparison of changes between the subject's tissue provides a method by which a health state of the subject's tissue can be predicted.

In one aspect, the method combines spatial and temporal processing to emphasize subtle temporal changes in a video. The method decomposes the video sequence into different spatial frequency bands. These bands might be magnified differently because (a) they might exhibit different signal-to-noise ratios or (b) they might contain spatial frequencies for which the linear approximation used in motion magnification does not hold. In the latter case, the method reduces the amplification for these bands to suppress artifacts. When the goal of spatial processing is to increase temporal signal-to-noise ratio by pooling multiple pixels, the method spatially low-pass filters the frames of the video and downsamples them for computational efficiency. In the general case, however, the method computes a full Laplacian pyramid.

The method then performs temporal processing on each spatial band. The method considers the time series corresponding to the value of a pixel in a frequency band and applies a bandpass filter to extract the frequency bands of interest. As one example, the method can select frequencies within the range of 0.4-4 Hz, corresponding to 24-240 beats per minute, if the user wants to magnify a pulse associated with the living tissue, for example. If the method extracts the pulse rate, it can employ a narrow frequency band around that value. The temporal processing is uniform for all spatial levels and for all pixels within each level. The method then multiplies the extracted bandpassed signal by a magnification factor α. This factor can be specified by the user, and can be attenuated automatically. Next, the method adds the magnified signal to the original signal and collapses the spatial pyramid to obtain the final output. Since natural videos are spatially and temporally smooth, and since the filtering is performed uniformly over the pixels, the method implicitly maintains spatio-temporal coherency of the results. The present method can amplify small motion without tracking motion as in Lagrangian methods. Temporal processing produces motion magnification using an analysis that relies on the first-order Taylor series expansions common in optical flow analyses as explained in U.S. Pub. 2014/0072190 to Wu et al. which is incorporated herein by reference in its entirety.

To process an input image by Eulerian video magnification, a user (or pre-programmed processor) can (1) select a temporal bandpass filter; (2) select an amplification factor, a; (3) select a spatial frequency cutoff (specified by spatial wavelength, λ_{c}) beyond which an attenuated version of α is used; and (4) select the form of the attenuation for α—either force a to zero for all λ<λ_{c}, or linearly scale a down to zero. The frequency band of interest can be chosen automatically in some cases, but it is often important for users to be able to control the frequency band corresponding to their application. In a real-time application, the amplification factor and cutoff frequencies are all customizable by the user.

In one aspect of the technology, the camera assets described herein can be configured to detect wavelengths of light in a variety of wavelengths of light. For example, in one aspect, the camera can be configured to detect a first band of wavelengths of light ranging from approximately 150 to 400 nm, a second band of wavelengths of light ranging from approximately 400 to 700 nm, and a third band of wavelengths of light ranging from approximately 700 to 1100 nm. Advantageously, data regarding the subject's tissue state which may not be observable in the conventional visible spectrum of light (i.e., 400 to 700 nm) can be observed and used in connection with predicting the tissue state of the subject. A light source can be associated with the camera configured to propagate a wavelength of light onto the tissue of the subject. In one aspect, the light source can be capable of propagating a single wavelength of light or a band of wavelengths of light ranging from approximately 150 to 400 nm, approximately 400 to 700 nm, and approximately 700 to 1100 nm. Certain micro-variations in the coloration and/or size of tissues are enhanced when subject to different wavelengths of light. Accordingly, for different diseases of interest, the image of the tissue can be captured under wavelengths of light that optimize detection of the disease. In aspects of the technology, a contrasting agent can be used. Where previous methods have employed contrasting agents, the technology disclosed herein can be capable of detecting micro-variations in the tissues responding to contrasting agents that were previously difficult to detect with the naked or unaided eye.

In one aspect, Eulerian video magnification can be used to amplify subtle motions of blood vessels in tissues (e.g., a radial artery and an ulnar artery) arising from blood flow. In this aspect, the temporal filter can be tuned to a frequency band that includes the heart rate (e.g., 0.88 Hz (53 bpm)) and the amplification factor can be set to α=10. To reduce motion magnification of irrelevant objects, a user-given mask amplifies the area near the wrist only. Movement of the radial artery and the ulnar artery can barely be seen in an unprocessed input video, but is significantly more noticeable in the motion-magnified output. While more noticeable to the naked eye, the motion is more pronounced and hence more useable in detecting and diagnosing changes in the state of the subject's tissue.

In one aspect of the technology, the process selects the temporal bandpass filter to pull out the motions or signals to be amplified. The choice of filter is generally application dependent. For motion magnification, a filter with a broad passband is preferred; for color amplification of blood flow, a narrow passband produces a more noise-free result. Ideal bandpass filters are used for color amplification, since they have passbands with sharp cutoff frequencies. Low-order IIR filters can be useful for both color amplification and motion magnification and are convenient for a real-time implementation. In general, two first-order lowpass IIR filters with cutoff frequencies ω₁ and ωₕ can be used to construct an IIR bandpass filter. The process selects the desired magnification value, α, and spatial frequency cutoff, λ_{c}. Various α and λ_{c} values can be used to achieve a desired result. The user can select a higher α value that violates the band to exaggerate specific motions or color changes at the cost of increasing noise or introducing more artifacts. In one aspect of the technology, the Eularian motion magnification can confirm the accuracy of a heart rate estimate and can verify that the color amplification signal extracted from the method matches the photoplethysmogram, an optically obtained measurement of the perfusion of blood to the skin, as measured by the monitor.

The method takes a video as input and exaggerates subtle color changes and micro-motions. To amplify motion, the method does not perform feature tracking or optical flow computation, but magnifies temporal color changes using spatio-temporal processing. This Eulerian based method, which temporally processes pixels in a fixed spatial region, successfully reveals informative signals and amplifies small motions in real-world videos. The Eulerian based method begins by examining pixel values of two or more images. The method then determines the temporal variation of the examined pixel values. The method is designed to amplify only small temporal variations. While the method can be applied to large temporal variations, the advantage in the method is provided for small temporal variations. Therefore, the method is optimized when the input video has small temporal variations between the images. The method then applies signal processing to the pixel values. For example, signal processing can amplify the determined temporal variations, even when the temporal variations are small.

In one non-limiting example, the current technology is employed to corroborate and/or replace a PET scan. Positron emission tomography (PET) is a nuclear medicine, functional imaging technique that produces a three-dimensional image of functional processes in the body. The system detects pairs of gamma rays emitted indirectly by a positron-emitting radionuclide (tracer), which is introduced into the body on a biologically active molecule. Three-dimensional images of tracer concentration within the body are then constructed by computer analysis. If the biologically active molecule chosen for PET is fluorodeoxyglucose (FDG), for example, the concentrations of tracers imaged will indicate tissue metabolic activity as it corresponds to the regional glucose uptake. Use of this tracer to explore the possibility of cancer metastasis (i.e., spreading to other sites) is a common type of PET scan in standard medical care. In aspects of the current technology, the estimation of blood flow (or lack thereof) and micro-variations in the size and/or coloration of tissue are used to indicate tissue metabolic activity. Rather than image radioactivity of tracers, it is believed that amplified micro-variations in coloration of tissues corresponding to regional glucose uptake suggests metabolic activity that is similarly collected from a PET scan. By correlating micro-variations in the coloration of tissues with known data collected from PET scans, a pathology profile is generated for use in future comparisons to predict diseased states of tissues.

In another aspect of the technology, previously undetected responses to tissue excitation from light energy are amplified to generate a tissue pathology profile, both for a subject as well as third-party correlation data. For example, it is believed that tissues having a certain diseased state will fluoresce and/or react to excitement from certain wavelengths of light propagated onto the tissue of the subject. In one aspect, tissues that are in a particular diseased state will scatter and/or absorb light at a certain frequency. Rather than relying on “naked eye” observations to assess the tissue reaction to the wavelength(s) of light, micro-variations are amplified by way of the current process to develop a pathology profile of the tissue. The specific optical spectrum of a tissue sample contains information about the biochemical composition and/or the structure of the tissue. The biochemical information can be obtained by measuring absorption, fluorescence, or Raman scattering signals. Structural and morphological information can be obtained by techniques that look at the elastic-scattering properties of tissue. It is believed that the amplified images from these approaches are useful for the detection of cancer as well as for other diagnostic applications (e.g., blood oxygen saturation, intra-luminal detection of atherosclerosis, etc.) and simply the identification of different tissue types during procedures.

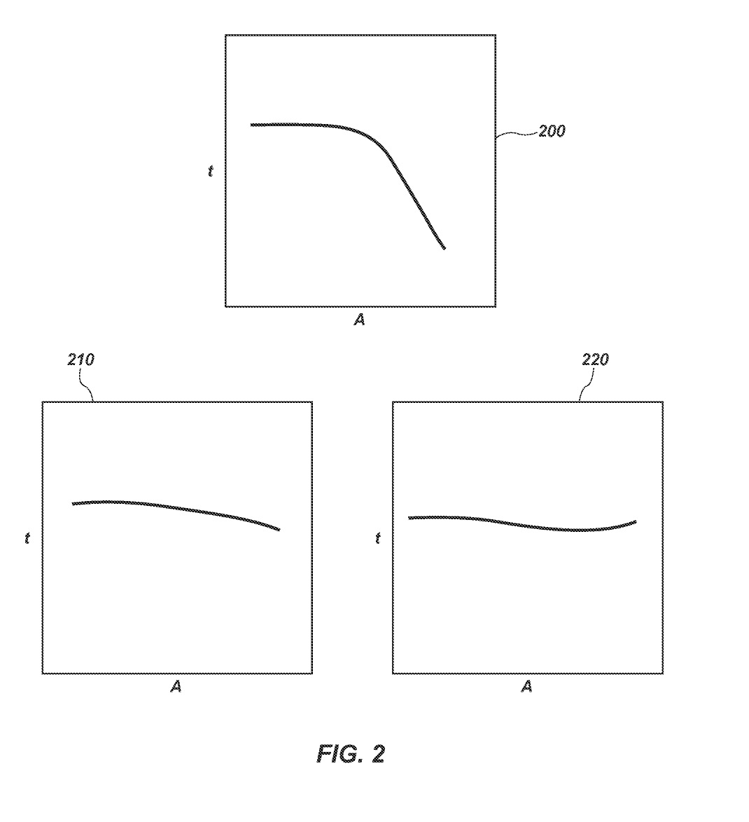

In one aspect of the technology, datasets of baseline data related to subject tissues are collected and a profile of subject characteristics is generated. For example, a profile of a subject's tissue includes a plurality of images (video or still) of the subject's skin (taken for example with the subject's mobile phone) throughout the day during the subject's normal activities. The subject's temperature, heartrate, and other related health meta data can also be included in the profile. FIG. 2 illustrates a graphical representation of a generic profile 200 generated for a microscopically detected change (delta) over time (t) of the coloration of a subject's living tissue (i.e., tissue that continues to remain on the subject). A graphical representation of a baseline coloration profile (i.e., an aggregation of historical data) for the same subject's tissue is shown on 210. A graphical representation of an aggregate third-party tissue coloration profile is presented at 220. In each representation, the change (delta) of the same microscopically detected change (e.g., skin coloration) over the same time (t) period is presented. While skin coloration is specifically referenced, it is understood that the pattern of blood flow through tissues, the change in shape of coloration, size of the tissue, texture of tissues, motion of tissues, etc. can all form part of the tissue profile of the subject.

Metadata associated with the subject's diet, sleeping patterns, and other activities can also be included in the profile. The subject's profile can be compared with his or her own profile in the past and used as a basis for determining the state of the subject's tissue. In another aspect of the technology, the subject's tissue profile can be compared with an aggregate profile of third-parties to discern the state of the subject's tissue. In this manner, outwardly observable micro-indicators can be correlated with third-party tissue states (e.g., healthy, diseased, at-risk, etc.) to predict the subject's own tissue state. In one aspect, the subject's own tissue profile can be that of a previous “normal” state (i.e., healthy) and/or a previous modified (i.e., not healthy) state.

In one aspect, aggregation includes normalization of a dataset limited by user-selected categories. Given a finite set of health-related categories, datasets can be grouped in categories. Non-limiting example categories can include blood analyses, MRI images, diseased states of tissue, medical history, medical prescriptions, family history, health habits, age, gender, weight, and/or race, and others as recognized by those skilled in the art. Aggregated groups can further be grouped into subclasses as suits a particular analysis. In one non-limiting example, an aggregate profile can be generated for the visual appearance of living tissues of female subjects suffering from melanoma and receiving chemotherapy. The aggregate profile can be used as a baseline comparison for a specific subject falling into the same category to determine the subject's deviation from or similarity to the aggregate profile. In each category, and where possible, data can be sorted by timeline. In addition, free text-based medical reports can be parsed and searched for medical concepts and related numerical entities extracted to be used in connection with the generation of aggregate profile data. Importantly, once a comparison has been made between an aggregate profile and a specific subject profile, the aggregate profile can be amended or modified to include the data of the specific subject profile. In this manner, the aggregate profile can be “evolving” with each measurement of a subject.

In one aspect of the technology, data can be transformed in that graphical displays, plots, charts of data, etc. can be generated. Humans are known to be able to absorb a lot of visual information in a very short time frame (50% of the cerebral cortex is for vision). To assist the practitioner, presenting the information graphically rather than textually allows the healthcare provider to absorb the information quickly. Information graphics (e.g., graphical displays, plots, charts, etc.) can thus be used to show statistics or evolution of these values over time.

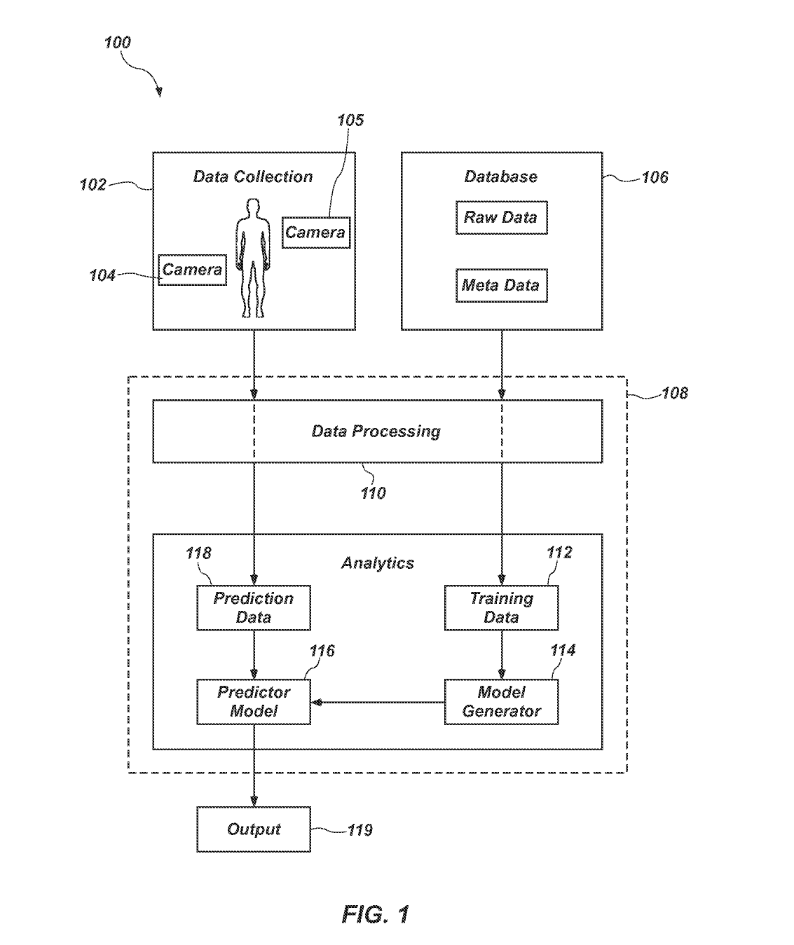

With reference to FIG. 1, a generalized architecture for the present technology includes a system 100 for analyzing outward variations or micro-variations of the tissues of a subject for diagnosis of the state of a subject (i.e., a diseased or healthy state of the tissue). Starting at box 102, one or more camera devices 104, 105 can be configured to capture images containing variations and particularly micro-variations in physiological conditions of the tissue of the subject. Each of the camera devices 104, 105 generates images comprising a time series of image values. In one aspect, the camera device comprises an image capture device, such as a charge-coupled device (CCD) camera or a complementary metal oxide semiconductor (CMOS) image sensor as known in the art. While camera devices are specifically referenced herein, any device capable of capturing an image can be used without departing from the scope of the technology described herein. For example, a plurality of CT scans or MRI images could be used to amplify micro-variations between the images themselves in order to assess the tissue of the subject. Moreover, while two camera devices are disclosed in FIG. 1, it is understood that a single camera device or more than two camera devices can be used to analyze tissues without departing from the scope of the technology.

Following the branch to the right of box 102, the output signals of camera devices 104, 105 can be sent to and stored in a memory component to create an archival database 106. Database 106 can house measurements of third-parties as well as the current subject. Database 106 can decode and store a segment of the raw data representing the signal from one or more cameras 104, 105 as well as meta data, which can include the subjects' (or third-parties′) demographic, including, but without limitation to, surname, gender, ethnicity, date of birth, weight, medical history, and so on. In addition, any information regarding the data collection system such as type, manufacturer, model, sensor ID, sampling frequency, and the like can also be stored. One or more data segments of interest can be communicated to a processor represented at 108. The processor 108 can be a computing device configured to obtain data generated by the camera devices 104, 105 and to perform calculations based on the obtained data. In one embodiment, the computing device can include at least one processor 108, an interface for coupling the computing device to the database 106, and a non-transitory computer-readable medium. The computer-readable medium can comprise computer-executable instructions stored thereon that, in response to execution by the processor 108, cause the processor 108 to perform the described calculations on the obtained data. One example of a suitable computing device can be a personal computer specifically programmed to perform the actions described herein. This example should not be taken as limiting, as any suitable computing device, such as a laptop computer, a smartphone, a tablet computer, a cloud computing platform, an embedded device, and the like, can be used in various embodiments of the present disclosure.

In one aspect of the technology, a camera device 104 can be coupled to a wearable device such as a watch, arm band, or chest strap that disposes the camera device 104 against a portion of the skin of the wearer (e.g., about the wrist of the subject). In one aspect, the camera device 104 can be worn such that a lens of the camera 104 is in contact with (or very near) the surface of the skin. A lighting device (e.g., LED, laser, etc.) can be coupled to the camera 104 and can be configured to propagate a beam of light onto the tissue of the subject. The beam of light can be directed to the tissue of the subject that is being imaged and analyzed. In one aspect, the processor 108 can be coupled to the device worn by the subject. In another aspect, however, image data collected by the camera device 104 can be transmitted to a remote processor 108 and data storage device for analysis. The straps (or other mechanical means known in the art) that couple the camera device 104 can be configured to secure the camera device 104 firmly against the tissue of the subject being analyzed, such that the area being analyzed is not inadvertently substantially changed. In the aspect where the camera device 104 is coupled to a device worn about the wrist of the subject, the tissue is analyzed more for general changes in the subjects overall physiological state. That is, in one non-limiting example, the tissue is analyzed to determine a baseline, and the rate of change of heart-rate, temperature, quantity and composition of sweat, blood pressure, blood oxygen level, blood flow rate, etc. These characteristics can be used as a comparison data point with pre-existing baseline data of the subject and/or compared to third-party data that corresponds to known “normal” or “diseased” states of the body.

In another aspect of the technology, a camera device 104 can be coupled to an elongate member configured to be placed within a cavity of a patient (e.g., an endoscopic device). In this aspect, the camera device 104 can be disposed about the distal end of the elongate member and used to image in-vivo tissues within the body of the patient. A lighting device (e.g., LED, laser, etc.) can be coupled to the camera 104 and can be configured to propagate a beam of light onto the in-vivo tissue of the subject. The beam of light can be directed to the tissue of the subject that is being imaged and analyzed. In one aspect, a processor 108 can be coupled directly to the elongate medical device. In another aspect, however, image data collected by the camera device 104 can be transmitted to a remote processor 108 and data storage device for further analysis. In an additional aspect, the camera device 104 can be coupled to a display configured to show a current image being captured by the camera device 104 as well as historical images, and images that have been amplified by the methods described herein to accentuate specific tissue characteristics.

With reference again to FIG. 1, as described in more detail below, the time segment of archived data can be preprocessed (box 110) to a form for further analyzing in accordance with the technology herein. The result is an altered dataset which can be referred to as “training data” (box 112) or “baseline data” retrieved from the subject. The training data can be used to create a model that indicates the correlation between the camera data from the archive and a state of the subject. In FIG. 1, model generation is represented at 114 and the resulting model stored in the computing device is represented at 116. Returning to box 102, once the model 116 has been generated, the camera devices 104, 105 can be coupled to the processor 108 by a real-time connection, such as by a serial cable, a USB cable, a local network connection, such as a Bluetooth connection, a wired local-area network connection, a WIFI connection, an infrared connection, and the like. In another embodiment, the camera devices 104, 105 can be coupled to the processor 108 by a wide area network, such as the Internet, a WiMAX network, a 3G network, a GSM network, and the like. The camera devices 104, 105 can each include network interface components that couple each camera device 104, 105 to the processor 108. Alternatively, the camera devices 104, 105 can each be coupled to a shared networking device via a direct physical connection or a local network connection, which in turn establishes a connection to the processor 108 over a wide area network.

The direct physical connection aspects and the local area network connection embodiments can be useful in a scenario when the camera devices 104, 105 are located in close proximity to the processor 108, such as within the same examination room. The wide area network embodiments can be useful in a larger tele-health or automated diagnosis application. In this branch (the real time branch) the signals from the camera devices 104, 105 are preprocessed to the same format as the archived data during model generation 114, resulting in “prediction data.” Ultimately the signal processing device uses the model to examine the prediction data and provide an output of a prediction of a state of a subject that is found to be correlated to the input from the camera(s) based on the training data (or aggregate third-party data). The states of the subject with which the present technology is concerned are those for which the correlation with the outwardly manifested micro-physiological data is established and modeled as described above. However, the linking of known states of the subject can also be taken into consideration (i.e., lack of sleep, poor diet, lack of water, history of hypoglycemia, etc.). Depending on the event and the established relationship, the output can be binary (yes/no) or have more than two digital quantities to indicate a predictive probability or a degree of presence or severity. The output can be on a display or by means of a signal, for example.

### Prediction of States of Tissue and Subject

In one aspect of the technology, the correlation of the outwardly manifested micro-data with the occurrence of the state (e.g., cancer, ischemia, stressed, normal, etc.) of a subject's tissue can be established by a multiple regression analysis. For the analysis, let Y represent a dependent or criterion variable indicative of the tissue state of interest, and let X1, X2, X3, . . . , Xn represent independent or predictor variables (i.e., the data derived from the camera) of Y. An observation of Y coupled with observations of the independent variables Xi is a case or a run of an experiment. Typically, observations of values for any given variable will form a continuous, totally-ordered set. In cases where a variable is categorical or probabilistic (such as a 0 or 1 representing presence or absence of a medical condition) a logistic function can be used to represent the regression model. In experimental runs, score values of these variables are observed from a population. It is assumed that any dataset used is a sample from a population or larger group. Regression can be used to predict time series values of the dependent variable Y based on time series data of the independent variable X. Ideally, time series data for X will be sampled at regular intervals and will be represented by the Xi. Time series data for the dependent variable Y need not be sampled regularly. Observations of Yi and Xi will be made over a time period 0<t<T. Causality is assumed, and if Yt exists, Xt, Xt−1, 4 t−2, Xt−3, . . . X0 can be used in a multiple regression to predict it.

In accordance with one aspect of the technology, the predictor micro-variation of the tissue is sampled to obtain N samples between time t−N and time t. A spectral analysis (FFT in an example embodiment) can be used to obtain the waveform frequency components which are used in the multiple regression analysis. Another variable for the multiple regression analysis can be an indicator of the state of the subject's tissue at time t. This can be a binary indicator of a harmful medical condition indicating that the condition is likely present or absent, for example. The various observations are used in the multiple regression to set the values of the various coefficients of the predictors in the linear function. In one aspect, the predictor values can be the spectral components of the predictor signal. The result is the model that will reside in the processor. The processor derives the time lagged, spectrum analyzed predictor data signal from data processed from the camera device and uses the processor and the model to provide the output that indicates the prediction of the state (healthy, diseased, at-risk, etc.) of the subject's tissue. As distributed time-lagged regression can be performed on the data, the time scales of the alleged correlations between the two waveforms can be much longer than their sampling frequencies, and it can be desirable to manage the number of predictors. The predictors need to cover the time-lag region in which the suspected correlation is in place.

It is believed that use of spectral information (e.g., FFT) requires the use of many predictors in the model for the bandwidths of signals in use. However, multiple regression often benefits when less predictors can be used. The goal of reducing the independent variable set can be achieved when representative predictors are used, and when predictors can be placed in groups with similar characteristics. The placement of predictors into similar groups (i.e., subclasses of aggregate datasets) in the present technology can be achieved by the use of a clustering algorithm. Clustering algorithms group sets of observations, usually according to a parameter k representing the desired number of clusters to be found by the algorithm. Hierarchical clustering algorithms solve the clustering problem for all values of k using bottom up and top down methods. One suitable hierarchical clustering algorithm that can be used is called AGNES (see L. Kaufman and P. J. Rousseeuw. Finding Groups in Data, An Introduction to Cluster Analysis, Hoboken, N.J., Wiley-Interscience, 2005, which is hereby expressly incorporated by reference herein) to cluster the spectral predictors based on three criteria obtained from a multiple regression performed on the FFT coefficients. As measures of similarity used in clustering, these criteria are the FFT index, the regression coefficient estimates themselves, and the regression coefficient t values.

In one aspect of the technology, client computer(s)/devices and server computer(s) provide processing, storage, and input/output devices executing application programs and the like for use of the methods and processes described herein. Client computer(s)/devices can also be linked through communications network to other computing devices, including other client devices/processes and server computer(s). Communications network can be part of a remote access network, a global network (e.g., the Internet), a worldwide collection of computers, local area or wide area networks, and gateways that currently use respective protocols (TCP/IP, Bluetooth, etc.) to communicate with one another. Other electronic device/computer network architectures are suitable.

In accordance with one aspect of the technology, a computer contains a system bus, where a bus can be a set of hardware lines used for data transfer among the components of a computer or processing system. The bus can essentially be a shared conduit that connects different elements of a computer system (e.g., processor, disk storage, memory, input/output ports, network ports, etc.) that enables the transfer of information between the elements. Attached to the system bus can be an I/O device interface for connecting various input and output devices (e.g., keyboard, mouse, displays, printers, speakers, etc.) to the computer. A network interface allows the computer to connect to various other devices attached to a network. A memory provides volatile storage for computer software instructions and data used to implement an embodiment of the present invention (e.g., code detailed above). A disk storage provides non-volatile storage for computer software instructions and data used to implement an embodiment of the present invention. A central processor unit can also be attached to the system bus and provides for the execution of computer instructions.

In one embodiment, the processor routines and data are a computer program product, including a computer readable medium (e.g., a removable storage medium such as one or more DVD-ROM's, CD-ROM's, diskettes, tapes, etc.) that provides at least a portion of the software instructions for the invention system. A computer program product can be installed by any suitable software installation procedure, as is well known in the art. In another aspect, at least a portion of the software instructions can also be downloaded over a cable, communication and/or wireless connection. In other aspects, the programs comprise a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present technology. In alternate aspects, the propagated signal can be an analog carrier wave or digital signal carried on the propagated medium. For example, the propagated signal can be a digitized signal propagated over a global network (e.g., the Internet), a telecommunications network, or other network. In one embodiment, the propagated signal can be a signal that is transmitted over the propagation medium over a period of time, such as the instructions for a software application sent in packets over a network over a period of milliseconds, seconds, minutes, or longer. In another embodiment, the computer readable medium of computer program product can be a propagation medium that the computer system can receive and read, such as by receiving the propagation medium and identifying a propagated signal embodied in the propagation medium, as described above for computer program propagated signal product. Generally speaking, the term “carrier medium” or transient carrier encompasses the foregoing transient signals, propagated signals, propagated medium, storage medium and the like.

The foregoing detailed description describes the technology with reference to specific exemplary aspects. However, it will be appreciated that various modifications and changes can be made without departing from the scope of the present technology as set forth in the appended claims. The detailed description and accompanying drawings are to be regarded as merely illustrative, rather than as restrictive, and all such modifications or changes, if any, are intended to fall within the scope of the present technology as described and set forth herein.

More specifically, while illustrative exemplary aspects of the technology have been described herein, the present technology is not limited to these aspects, but includes any and all aspects having modifications, omissions, combinations (e.g., of aspects across various aspects), adaptations and/or alterations as would be appreciated by those skilled in the art based on the foregoing detailed description. The limitations in the claims are to be interpreted broadly based on the language employed in the claims and not limited to examples described in the foregoing detailed description or during the prosecution of the application, which examples are to be construed as non-exclusive. For example, in the present disclosure, the term “preferably” is non-exclusive where it is intended to mean “preferably, but not limited to.” Any steps recited in any method or process claims can be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus-function are expressly recited in the description herein. Accordingly, the scope of the technology should be determined solely by the appended claims and their legal equivalents, rather than by the descriptions and examples given above.

1. A wearable device configured to monitor a physiological condition of a subject, comprising:
a wearable camera having a coupling member configured to attach the camera to a portion of the subject, the camera configured to capture a plurality of images of a portion of a tissue of the subject;
a processor in communication with the camera, said processor comprising executable code configured to amplify microscopic temporal variations between the plurality of images of the tissue of the subject and generate a profile of at least one microscopic temporally detected physiological variation of the tissue of the subject and store the profile in a database; and
wherein the processor is further configured to compare the profile of the tissue of the subject with a database corresponding to previous profiles of the at least one microscopic temporally detected physiological variation of the tissue of the subject.

2. The device of claim 1, wherein the processor is further configured to compare the profile of the tissue of the subject to an aggregate profile of a first plurality of third-party subjects, said aggregate profile of the first plurality of third-party subjects corresponding to the at least one microscopic temporally detected physiological variation of tissues of the first plurality of third-party subjects, the tissues of the first plurality of third-party subjects having a normal health state; and
wherein the processor is further configured to compare the profile of the tissue of the subject to an aggregate profile of a second plurality of third-party subjects, said aggregate profile of the second plurality of third-party subjects corresponding to the at least one microscopic temporally detected physiological variation of tissues of the second plurality of third-party subjects, the tissues of the second plurality of third-party subjects having a known diseased state; and
wherein said processor is further configured to detect differences between the profile of the tissue of the subject and the aggregate profile of the first plurality of third-party subjects and the aggregate profile of the second plurality of third-party subjects and determine a probability that a state of the subject's tissue corresponds to the diseased state of the tissues of the second plurality of third-party subjects.

3. The device of claim 1, wherein the physiological variations of the tissue comprise color and/or motion.

4. The device of claim 1, wherein the camera is configured to detect a first band of wavelengths of light ranging from approximately 150 to 400 nm, a second band of wavelengths of light ranging from approximately 400 to 700 nm, and a third band of wavelengths of light ranging from approximately 700 to 1100 nm.

5. The device of claim 4, further comprising a light source configured to propagate a beam of light comprising a first beam of light ranging from approximately 150 to 400 nm, a second beam of light ranging from approximately 400 to 700 nm, and a third beam of light ranging from approximately 700 to 1100 nm.

6. The device of claim 2, further comprising a remote database accessible by the wearable device, said database containing the plurality of aggregate profiles of the first and second plurality of third-party subjects corresponding to a plurality of disease states and non-disease states of the tissues of the first and second plurality of third-party subjects.

7. The device of claim 6, wherein the processor is further configured to wirelessly communicate with and access data from the database.

8. The device of claim 7, wherein the processor is configured to communicate with a remote computer device corresponding to a health care professional.

9. The device of claim 2, wherein the aggregate profiles of the first and second plurality of third-party subjects is restricted to one or more of the subjects' age, gender, race, weight, disease state, geographic location, altitude, season, or medications taken by the third-party subjects.

10. A device configured for in-vivo monitoring of the tissue of a subject, comprising:
an elongate medical device configured for placement into a portion of a body of the subject;
a camera disposed about a distal end of the elongate medical device, the camera configured to capture a plurality of images of tissue within the body of the subject;
a processor coupled to the camera, said processor comprising executable code configured to amplify microscopic temporal variations between the plurality of images of the tissue of the subject and generate a profile of at least one microscopic temporally detected physiological variation of the tissue of the subject;
wherein said processor is further configured to compare the profile of the tissue of the subject to an aggregate profile of a first plurality of third-party subjects, said aggregate profile of the first plurality of third-party subjects corresponding to the at least one microscopic temporally detected physiological variation of tissues of the first plurality of third-party subjects, the tissues of the first plurality of third-party subjects having a normal health state;
wherein the processor is further configured to compare the profile of the tissue of the subject to an aggregate profile of a second plurality of third-party subjects, said aggregate profile of the second plurality of third-party subjects corresponding to the at least one microscopic temporally detected physiological variation of tissues of the second plurality of third-party subjects, the tissues of the second plurality of third-party subjects having a known diseased state; and
wherein said processor is further configured to detect differences between the profile of the tissue of the subject and the aggregate profile of the first plurality of third-party subjects and the aggregate profile of the second plurality of third-party subjects and determine a probability that a state of the subject's tissue corresponds to the diseased state of the tissues of the second plurality of third-party subjects.

11. The device of claim 10, wherein the physiological variation comprises variations in the color of the tissue of the subject.

12. The device of claim 10, wherein the camera is configured to detect a first band of wavelengths of light ranging from approximately 150 to 400 nm, a second band of wavelengths of light ranging from approximately 400 to 700 nm, and/or a third band of wavelengths of light ranging from approximately 700 to 1100 nm.

13. The device of claim 12, further comprising a light source disposed about the distal end of the elongate medical device configured to propagate a beam of light comprising a first beam of light ranging from approximately 150 to 400 nm, a second beam of light ranging from approximately 400 to 700 nm, or a third beam of light ranging from approximately 700 to 1100 nm.

14. A non-destructive method for predicting diseased states of live tissues through optical measurements, comprising:
positioning a camera about an area of live tissue of a subject, wherein said camera is in communication with a processor configured to receive and process image data of the tissue, said processor comprising executable code configured to amplify microscopic temporal variations between a plurality of images of the tissue and generate a profile of at least one microscopic temporally detected physiological variation of the tissue;
receiving image data of the tissue through the camera and amplifying microscopic temporal variations between the plurality of images of the tissue;
generating a profile of at least one microscopic temporally detected physiological variation of the tissue;
comparing the profile of the live tissue to an aggregate profile of a first plurality of live tissues of third-party subjects, said aggregate profile of the first plurality of the third-party subjects corresponding to the at least one microscopic temporally detected physiological variation of live tissues of the first plurality of third-party subjects, said live tissues of the first plurality of third-party subjects having a normal health state;
comparing the profile of the live tissue to an aggregate profile of a second plurality of live tissues of third-party subjects, said aggregate profile of the second plurality of third-party subjects corresponding to the at least one microscopic temporally detected physiological variation of live tissues of the second plurality of third-party subjects, said live tissues of the second plurality of third-party subjects having a known diseased state; and
determining a probability that the live tissue of the subject corresponds to the diseased state of the live tissues of the second plurality of third-party subjects.

15. The method of claim 14, wherein the camera is attached to a coupling device configured to be removably fixed to a portion of the body of the subject.

16. The method of claim 15, wherein the camera is coupled to a light source configured to propagate light onto the tissue of the subject.

17. The method of claim 15, wherein the camera and light source are disposed adjacent of the tissue of the subject.

18. The method of claim 15, wherein the camera and light source are disposed about a device configured to be worn about the wrist of the subject.

19. The method of claim 14, wherein the camera is fixed to a distal end of an elongate member, the elongate member configured for placement into a body cavity of the subject.

20. The method of claim 19, wherein the camera is coupled to a light source configured to propagate light onto tissues within the body cavity of the subject.

21. The method of claim 14, further comprising generating a profile of changes to the microscopic temporally detected physiological variation of tissues over a predetermined period of time.

22. The method of claim 14, further comprising amplifying a plurality of microscopic temporal variations between the plurality of images of the tissue.

23. The method of claim 22, wherein the plurality of microscopic temporal variations comprise color of the tissue and motion of the tissue.

24. The method of claim 23, further comprising determining an estimate of physiological characteristics of the subject based on the amplified microscopic temporal variations between the plurality of images of the tissue.

25. The method of claim 24, wherein the physiological characteristics of the subject comprise pulse, blood pressure, breathing rate, temperature, or blood oxygen content.
